(19) United States
(12) Patent Application Publication
(10) Pub. No.: US 2011/0091905 A1
(43) Pub. Date: Apr. 21, 2011

(54) BIOCHIP AND METHOD OF DETECTING REACTION FROM THE SAME

(75) Inventors: Chil Seong AH, Daejeon (KR); Gun Yong Sung, Daejeon (KR); Wan Joong Kim, Goyang (KR); Bong Kyu Kim, Daejeon (KR); Jong Cheol Hong, Daejeon (KR); Chul Huh, Daejeon (KR); Seon Hee Park, Daejeon (KR)

(73) Assignee: Electronics and Telecommunications Research Institute, Daejeon (KR)

(21) Appl. No.: 12/893,166

(22) Filed: Sep. 29, 2010

(30) Foreign Application Priority Data
Oct. 15, 2009 (KR) 10-2009-0098016

(51) Int. Cl. G01N 33/53; G01N 21/17; C12M 1/34; G01N 30/00

(52) U.S. Cl. 435/7.8; 436/164; 435/288.7; 422/69

(57) ABSTRACT

Provided are a biochip and a method of detecting a reaction from the biochip. This method includes preparing a first mixture solution of polyvinylpyrrolidone (PVP) and a sample including target molecules, measuring absorbance or transmittance of the first mixture solution, preparing a second mixture solution including the PVP, the sample, and a receptor of the target molecules, measuring absorbance or transmittance of the second mixture solution, and calculating an absorbance or transmittance difference between the first mixture solution and the second mixture solution. Thus, it is possible to reduce the production cost of the biochip by inducing a reaction of an antigen and an antibody using PVP. Further, it is possible to detect an accurate quantity of the antigen by analyzing a quantity of antigen on the basis of the absorbance or transmittance difference.

### CROSS-REFERENCE TO RELATED APPLICATION

This application claims priority to and the benefit of Korean Patent Application No. 10-2009-0098016, filed Oct. 15, 2009, the disclosure of which is incorporated herein by reference in its entirety.

### BACKGROUND

1. Field of the Invention

The present invention relates to a biochip and, more particularly, to a method of detecting a reaction from a biochip using polyvinylpyrrolidone (PVP).

2. Discussion of Related Art

In general, analysis biochips test a variety of items such as occult blood, bilirubin, urobilinogen, ketone, protein, nitrite, glucose, pH, specific gravity, white blood cells, vitamin C, and so on.

Urinalysis using test paper is a semi-quantitative test that primarily screens and tests various diseases of the human body and can test abnormality of the human body in its early stage. Since urine sampling is easy, it is no burden to a testee, and its results can be immediately determined, this urinalysis is very high in utility. The urinalysis biochips display test results to the testees such that the testees can visually check abnormality of the aforementioned relevant items. However, because such biochips by nature use a test part corresponding to each test item attached to a plastic film, they have drawbacks in that it is difficult for the testees to visually discriminate a change in color shown as test results, and in that test accuracy may be lowered, for instance the visual discrimination may vary depending on an individual testee. Further, the high-accuracy tests must make use of expensive equipment, and be carried out by a specially educated expert, so that they require much time and cost.

Meanwhile, these biochips mainly use polyethylene glycol (PEG) as an induction agent for inducing a reaction of an antigen and an antibody. However, PEG is expensive, and thus the price of the biochip is raised.

### SUMMARY OF THE INVENTION

The present invention is directed to a method of detecting a reaction from a biochip capable of inducing a reaction of an antigen and an antibody without using polyethylene glycol (PEG), and a portable compact detecting apparatus based on an optical technique.

An aspect of the present invention provides a biochip, which analyzes a quantity of target molecules based on absorbance or transmittance of a mixture solution of polyvinylpyrrolidone (PVP) inducing a reaction; a sample including the target molecules; and a receptor reacting with the target molecules.

In exemplary embodiments, the absorbance or transmittance of the mixture solution may be determined by irradiating light onto the mixture solution and measuring the light absorbed or transmitted by the mixture solution.

In exemplary embodiments, the absorbance of the mixture solution may be measured by converting light irradiated onto the mixture solution and light absorbed or transmitted by the mixture solution into electrical quantities.

In exemplary embodiments, the quantity of target molecules may be analyzed by a difference between the absorbance or transmittance of the mixture solution and reference absorbance or transmittance defined by absorbance or transmittance of a mixture solution of the PVP and the sample.

Another aspect of the present invention provides a method of detecting a reaction from a biochip, which comprises: preparing a first mixture solution of polyvinylpyrrolidone (PVP) and a sample including target molecules; measuring absorbance or transmittance of the first mixture solution; preparing a second mixture solution including PVP, the sample, and a receptor of the target molecules; measuring absorbance or transmittance of the second mixture solution; and calculating an absorbance or transmittance difference between the first mixture solution and the second mixture solution.

In exemplary embodiments, the PVP and the sample of the first mixture solution may have the same concentrations as those of the second mixture solution.

In exemplary embodiments, measuring the absorbance or transmittance of the first or second mixture solution may include: applying a predetermined wavelength of light; causing the light to transmit the mixture solution; and calculating the absorbance or transmittance based on the transmitted light.

In exemplary embodiments, applying the light may include measuring the absorbance or transmittance of the first or second mixture solution according to wavelength.

In exemplary embodiments, the light may be irradiated from three color light sources.

In exemplary embodiments, the light transmitting the mixture solution may be received by a light receiving element including a photo diode.

In exemplary embodiments, the sample may be selected from urine, blood, and saliva.

In exemplary embodiments, the absorbance or transmittance difference between the first mixture solution and the second mixture solution may be proportional to the concentration of the target molecules.

### BRIEF DESCRIPTION OF THE DRAWINGS

The above and other features and advantages of the present invention will become more apparent to those of ordinary skill in the art by describing in detail preferred embodiments thereof with reference to the attached drawings in which:

FIG. 1 illustrates the configuration of a digital reader for urinalysis according to an exemplary embodiment of the present invention;

FIG. 2 is a flowchart illustrating a method of detecting a reaction from a biochip according to an exemplary embodiment of the present invention;

FIG. 3 is a graph showing absorbance according to wavelength in an exemplary embodiment of the present invention; and

FIGS. 4A through 4C are graphs showing absorbance according to the concentration of an antigen at different wavelengths

### DETAILED DESCRIPTION OF EXEMPLARY EMBODIMENTS

The present invention will be described more fully hereinafter with reference to the accompanying drawings, in which exemplary embodiments of the invention are shown. This invention may, however, be embodied in different forms and should not be construed as limited to the embodiments set forth herein. In the following description of the present invention, a detailed description of known functions and components incorporated herein will be omitted when it may make the subject matter of the present invention rather unclear. It should be noted that the same reference numbers are used in the figures to denote the same elements.

It will be understood that, throughout the specification, unless explicitly described to the contrary, the term “comprise” and its conjugations such as “comprises” or comprising” should be interpreted to include stated elements but not exclude any other elements. In addition, the term “section,” “device,” or “module” used herein refers to a unit for processing at least one of a function and an operation, which can be realized by hardware, software, or a combination thereof.

FIG. 1 illustrates the configuration of a digital reader for urinalysis according to an exemplary embodiment of the present invention.

Referring to FIG. 1, a urinalysis digital reader according to an exemplary embodiment of the present invention includes a light emitter 130 having three color light sources, and a light receiver 140 which directly receives light from the light emitter 130 or receives light transmitted from the light emitter 130 to a biochip, and which converts the light into an electric signal (i.e. performs photoelectric conversion).

A body 100 is formed in a C shape, and thus a support 110 is taken into or out of a space between opposite lower and upper surfaces of the body 100.

The support 110 moves into the body 100 with a biochip 200 mounted thereon. The light emitter 130, the light receiver 140, a sidewall 160, and a display 120 displaying test results are disposed over the body 100.

The light emitter 130 is configured of a combination of three light emitting diodes (LEDs) of red, blue, and green.

The three color light source elements of the light emitter 130 may variously control a wavelength of light combined by individual control.

The light receiver 140 may be implemented using light receiving elements (sensors) such as photodiodes or phototriodes. These sensors are configured in an array, so that it is possible to secure sensitivity and easy mounting of the biochip.

The sidewall 160 is provided between the light emitter 130 and the light receiver 140 so as to efficiently discriminate the light.

Further, the urinalysis digital reader further includes an amplifier, an analog-to-digital converter (ADC), a micro control unit (MCU), and a telecommunicator.

The amplifier amplifies an electric signal received from the light receiver 140, and the ADC converts the amplified electric signal to generate a digital signal according to absorbance or transmittance of the biochip 200.

The MCU analyzes the digital signal, and thus the absorbance or transmittance of the biochip 200 on the basis of the wavelength.

The telecommunicator sends results read out by the MCU to a remote clinic such as a hospital or a public health center, and may include a radio frequency identification system (RFID) chip as a telecommunication module. In this case, the MCU records the results to the RFID chip. When intending to send the results to the remote clinic, a user reads the results from the RFID chip using a wired and/or wireless terminal on which an RFID reader is mounted, and sends them to a remote terminal.

Further, the urinalysis digital reader may include a fluid control module (not shown), which is configured to move, stop, and mix microfluids so as to make efficient analysis in the biochip 200.

The fluid control module includes channels capable of moving, mixing, and stopping relevant solutions in order to facilitate the analysis of a biological sample, storage tanks storing fluids, a pump transferring the fluids, a valve controlling the transfer of the fluids, and a mixer for fluid control. In order to move, stop, and mix the fluids, an electrostatic motor, a piezoelectric pump, and a variety of existing driving means using hydraulic or pneumatic pressure, and ultrasonic waves may be used.

Meanwhile, the biochip 200 is inserted into the support 110 having an elastic member installed on the urinalysis digital reader, and then is fixed in a recess formed in a lever when arriving at a designated position. Here, the biochip 200 is provided thereon with either a chip fixing structure coupled with a spring mounted on the urinalysis digital reader or a polymer layer having elasticity. As such, the chip fixing structure helps maintain a constant interval between the biochip and the urinalysis digital reader when the urine analysis is made, and allows the biochip 200 to be measured regardless of an external impact or fluctuation.

Now, an operation of the urinalysis digital reader having this configuration will be described.

When the biochip 200 is inserted into the urinalysis digital reader, the urinalysis digital reader is switched on, and thus a signal informing that the biochip 200 is inserted is applied to the MCU. Then, a specified wavelength of light is emitted by the light emitter 130, and a part of the emitted light is absorbed or transmitted on a test region of the biochip 200, and the other part is transmitted. The transmitted light is received by the light receiver 140.

The light receiver 140 directly receives the light signal or converts the light signal into an electrical signal. The electrical signal converted by the light receiver 140 is subjected to signal processing, and is analyzed by the MCU. The analyzed results are displayed through the display 120.

Here, the biochip 200 induces a reaction between an antigen and an antibody using polyvinylpyrrolidone (PVP) rather than polyethylene glycol (PEG).

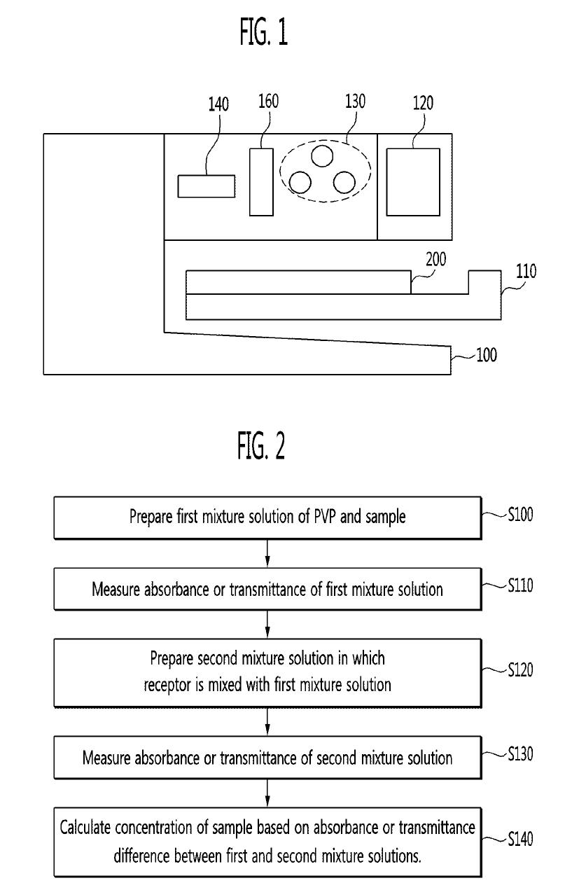

Hereinafter, detecting a reaction from the biochip using PVP will be described with reference to FIGS. 2 through 4C.

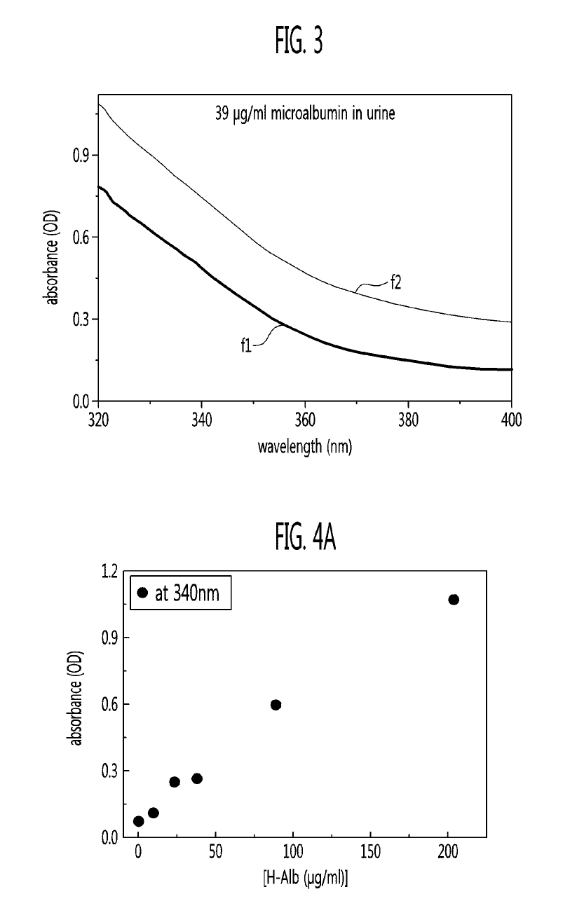

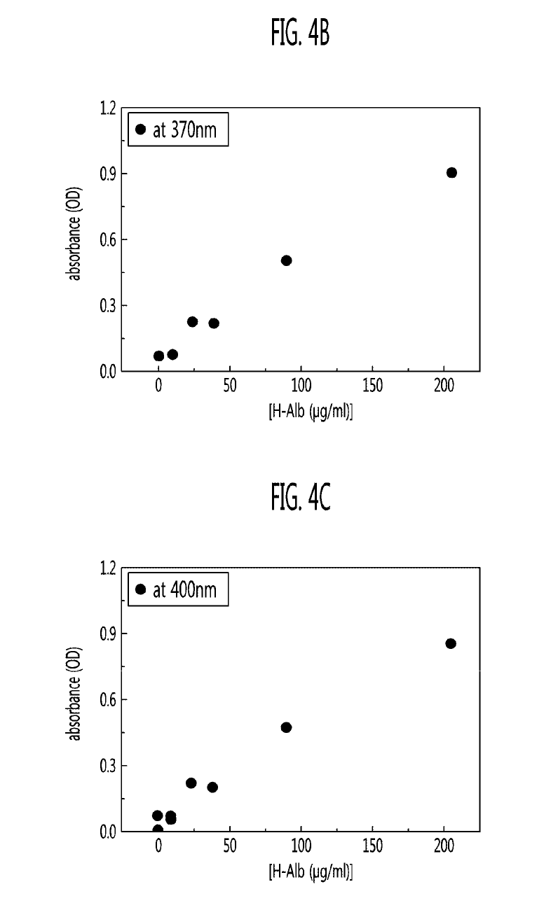

FIG. 2 is a flowchart explaining a method of detecting a reaction from a biochip according to an exemplary embodiment of the present invention, FIG. 3 is a graph showing absorbance according to wavelength in an exemplary embodiment of the present invention, and FIGS. 4A through 4C are graphs showing absorbance according to the concentration of an antigen at different wavelengths.

Referring to FIG. 2, first, a first mixture solution is prepared by mixture of PVP and a sample (antigen) (S100). The sample is a biological substance such as urine, blood, or saliva, and includes target molecules.

Next, the first mixture solution is supplied to a test region of the biochip 200, and the biochip 200 is left untouched for a predetermined time such that a reaction occurs.

When the predetermined time has lapsed, the biochip 200 is inserted into the urinalysis digital reader of FIG. 1, and light whose wavelength is adjusted is applied to the biochip 200.

At this time, the light receiver 140 receives the light transmitted from the biochip 200, the light signal detected by the light receiver 140 is calculated, and absorbance or transmittance of the first mixture solution is calculated (S110).

Here, an absorbance curve indicated by f1 of FIG. 3 is obtained. When the transmittance of light emitted from the light emitter 130 is detected according to wavelength of the light, the absorbance curve f1 shows that the longer the wavelength, the lower the absorbance. In the absorbance curve f1, the absorbance is obtained from the intensities of light I_{f10} and I_{f11} before and after the light of the light emitter 130 transmits the first mixture solution at a specified wavelength, respectively. The absorbance of the absorbance curve f1 is obtained by calculation of −log₁₀I_{f11}/I_{f10} (T (tansmittance)=I_{f11}/I_{f10}).

Next, a second mixture solution is prepared by mixture of PVP, a sample (antigen) and a receptor (antibody) (S120).

Here, the PVP and the sample (antigen) of the second mixture solution have the same concentration as those of the first mixture solution.

Next, the second mixture solution is supplied to the test region of the biochip 200, and the biochip 200 is left untouched for a predetermined time such that a reaction occurs. When the predetermined time has lapsed, the biochip 200 is inserted into the urinalysis digital reader of FIG. 1, and light is applied. A signal detected from the light receiver 140 is calculated, and absorbance or transmittance of the second mixture solution is calculated (S130). Thereby, an absorbance curve indicated by f2 of FIG. 3 is obtained. In the absorbance curve f2, the absorbance is obtained from an intensity of light I_{f10} before the light of the light emitter 130 transmits the first mixture solution at a specified wavelength and an intensity of light I_{f21} after the light of the light emitter 130 transmits the second mixture solution at a specified wavelength. The absorbance of the absorbance curve f2 is obtained by calculation of −log₁₀I_{f21}/I_{f10} (T (tansmittance)=I_{f21} /I_{f10}).

It is assumed that the intensities of light of the absorbance curves f1 and f2 are I1 and I2, respectively. When an absorbance difference between the first and second mixture solutions with respect to each wavelength is calculated, it is given as I2−I2.

When the absorbance difference, I2-I1, is calculated according to the concentration of the sample as in FIGS. 4A through 4C, it can be found that as the concentration of the sample increases with respect to a specified wavelength of light, the absorbance increases.

The concentration of the sample can be accurately measured according to the absorbance difference calculated using this graph. This reaction is possible using PVP rather than PEG.

According to exemplary embodiments, it is possible to reduce the cost of production of the biochip by inducing a reaction of an antigen and an antibody using PVP. Further, it is possible to detect an accurate quantity of the antigen by analyzing a quantity of antigen on the basis of the absorbance or transmittance difference.

The exemplary embodiments of the present invention described above can be implemented not only by the apparatus and/or method, but by a program that achieves the function corresponding to the configuration of the exemplary embodiments of the present invention or a recording medium on which the program is recorded. This will be easily implemented from the disclosure of the aforementioned exemplary embodiments of the present invention by those skilled in the art.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A biochip analyzing a quantity of target molecules based on absorbance or transmittance of a mixture solution of:
polyvinylpyrrolidone (PVP) inducing a reaction;
a sample including the target molecules; and
a receptor reacting with the target molecules.

2. The biochip according to claim 1, wherein the absorbance or transmittance of the mixture solution is determined by irradiating light onto the mixture solution and measuring the light absorbed or transmitted by the mixture solution.

3. The biochip according to claim 1, wherein the absorbance or transmittance of the mixture solution is measured by converting light irradiated onto the mixture solution and light absorbed or transmitted by the mixture solution into electrical quantities.

4. The biochip according to claim 1, wherein the quantity of target molecules is analyzed by a difference between the absorbance or transmittance of the mixture solution and reference absorbance or transmittance defined by absorbance or transmittance of a mixture solution of the PVP and the sample.

5. A method of detecting a reaction from a biochip, comprising:
preparing a first mixture solution of polyvinylpyrrolidone (PVP) and a sample including target molecules;
measuring absorbance or transmittance of the first mixture solution;
preparing a second mixture solution including the PVP, the sample, and a receptor of the target molecules;
measuring absorbance or transmittance of the second mixture solution; and
calculating an absorbance or transmittance difference between the first mixture solution and the second mixture solution.

6. The method according to claim 5, wherein the PVP and the sample of the first mixture solution have the same concentrations as those of the second mixture solution.

7. The method according to claim 5, wherein measuring the absorbance or transmittance of the first or second mixture solution includes:
applying a predetermined wavelength of light;
causing the light to transmit the mixture solution; and
calculating the absorbance or transmittance based on the transmitted light.

8. The method according to claim 7, wherein applying the light includes measuring the absorbance or transmittance of the first or second mixture solution according to wavelength.

9. The method according to claim 7, wherein the light is irradiated from three color light sources.

10. The method according to claim 7, wherein the light transmitting the mixture solution is received by a light receiving element including a photo diode.

11. The method according to claim 5, wherein the sample is selected from urine, blood, and saliva.

12. The method according to claim 5, wherein the absorbance or transmittance difference between the first mixture solution and the second mixture solution is proportional to the concentration of the target molecules.
